(19) United States
(12) Patent Application Publication
(10) Pub. No.: US 2024/0172982 A1
(43) Pub. Date: May 30, 2024

(54) ELECTRONIC DEVICE WITH THREE-DIMENSIONAL NANOPROBE DEVICE

(71) Applicant: SAMSUNG ELECTRONICS CO., LTD., Suwon-si (KR)

(72) Inventors: Mikyung KIM, Suwon-si (KR); Kitae PARK, Suwon-si (KR); Jonghan KIM, Suwon-si (KR); Seong Joong KIM, Suwon-si (KR); Chisung BAE, Suwon-si (KR); Hyeokki HONG, Suwon-si (KR)

(73) Assignee: SAMSUNG ELECTRONICS CO., LTD., Suwon-si (KR)

(21) Appl. No.: 18/319,827

(22) Filed: May 18, 2023

(30) Foreign Application Priority Data
Nov. 24, 2022 (KR) 10-2022-0159621

(51) Int. Cl. A61B 5/25

(52) U.S. Cl. CPC A61B 5/25; A61B 2562/0285

(57) ABSTRACT

A three-dimensional (3D) nanoprobe device includes a body portion including a plurality of body layers that are stacked, a plurality of nanoprobes respectively extending longitudinally from the body portion, with each of the plurality of nanoprobes comprising a plurality of extension layers that are stacked, and an electrode portion disposed on a portion of the body portion.

### CROSS-REFERENCE TO RELATED APPLICATIONS

This application claims the benefit under 35 USC § 119(a) of Korean Patent Application No. 10-2022-0159621, filed on Nov. 24, 2022, in the Korean Intellectual Property Office, the entire disclosure of which is incorporated herein by reference for all purposes.

### BACKGROUND

### 1. Field

The following description relates to an electronic device with a three-dimensional (3D) nanoprobe device.

### 2. Background Art

In bio-healthcare fields, an implantable device may be designed to minimize damages and reactions in the body that may occur due to the implantation of a device. For example, an implantable device may include, for example, an electrode needle patch based on a nano/micro-scale tip structure. The implantable device may be inserted into a body to apply an external stimulus. In addition, the implantable device may enable extracellular or intracellular recording. The intracellular recording may be used to measure various signals, such as excitatory postsynaptic potential and inhibitory postsynaptic potential. The intracellular recording may include measuring the voltage or current of a cell membrane, e.g., an electrode may be in contact with an inner membrane of a cell. In addition, needles for such measurements may meet factors such as low invasiveness, high-spatial resolution, and high signal-to-noise.

The above description has been possessed or acquired by the inventor(s) in the course of conceiving the present disclosure and is not necessarily an art publicly known before the present application is filed.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one or more general aspects, a three-dimensional (3D) nanoprobe device includes a body portion including a plurality of body layers that are stacked, a plurality of nanoprobes respectively extending longitudinally from the body portion, with each of the plurality of nanoprobes comprising a plurality of extension layers that are stacked, and an electrode portion disposed on a portion of the body portion.

For two or more of the plurality of nanoprobes, the plurality of extension layers respectively may extend from, and may be respectively longitudinally continuous with, the body layers.

Each nanoprobe of the plurality of nanoprobes may be spaced apart from a nearest other nanoprobe of the plurality of nanoprobes by about 10 nanometers (nm) to about 100 millimeters (mm) in a transverse direction of the body portion.

The plurality of nanoprobes may have any one or any combination of any two or more of a thickness of about 10 nm to about 200 micrometers (μm), a width of about 10 nm to about 100 mm, or a length of about 0 nm to about 100 mm.

Each of two or more of the plurality of nanoprobes may have an aspect ratio of about 0.000005 to about 200000.

Each of two or more of the plurality of nanoprobes, may include a separation region respectively between two or more of the plurality of extension layers, and the separation region may be empty or at least a portion of the separation region may include a water-soluble polymer.

A portion of the separation region may be empty and another portion of the separation region may include the water-soluble polymer.

Respective tip regions of the two or more of the plurality of nanoprobes may be empty and another region of the separation region may include a water-soluble polymer.

The separation region may have a height of about 10 nm to about 30 μm.

For each of two or more of the nanoprobes, respective extension layers, of the two or more of the nanoprobes, may be sequentially stacked such that adjacent nanoprobes overlap each other, or may be cross-stacked such that the adjacent nanoprobes cross each other.

For each of two or more of the nanoprobes, respective extension layers of adjacent nanoprobes may be cross-stacked such that the adjacent nanoprobes are free of overlapping regions.

For each of two or more of the nanoprobes, respective extension layers of adjacent nanoprobes may be cross-stacked such that the adjacent nanoprobes overlap each other with an overlap width corresponding to 0% to less than 100% of an average width of the nanoprobes.

Each nanoprobe of the plurality of nanoprobes may include a base layer having a shape of the nanoprobe, an electrode pattern layer disposed on the base layer, and a protective layer, disposed on the electrode pattern layer, having an exposed tip region. The base layer extends from the body portion.

For each of two or more of the plurality of nanoprobes, the plurality of extension layers may include a base layer having a shape of a corresponding nanoprobe, an electrode pattern layer disposed on the base layer, a first protective layer disposed on the electrode pattern layer, an electrode pad layer disposed on the first protective layer, and a second protective layer disposed on the electrode pad layer. The base layer may extend from the body portion. A tip region of the corresponding nanoprobe may be exposed in the first protective layer and the second protective layer, and an electrode pad of the electrode pad layer may be exposed in the second protective layer.

For each of two or more of the plurality of nanoprobes, each of the base layer, the protective layer, the first protective layer, and the second protective layer may have a thickness of about 10 nm to about 200 μm.

The electrode pattern layer may include an electrode pattern including any one or any combination of any two or more of a straight line, an oblique line, a zigzag, a streamline, a curve, or a comb profile, and the electrode pad layer may include an electrode pad having a shape of any one or any combination of any two or more of a circle, an ellipse, a doughnut, or a polygon.

In another one or more aspects, an electronic device includes a three-dimensional (3D) nanoprobe device. The 3D nanoprobe includes a body portion in which a plurality of body layers is stacked, an extension portion extending longitudinally from the body portion and in which a plurality of extension layers having a plurality of nanoprobes is stacked, and an electrode portion disposed on a portion of the body portion.

The electronic device may be configured to measure any one or any combination of any two or more of a biosignal, a biomaterial, and a brain signal.

The electronic device may be configured to apply an external stimulus to a living body or be an implantable device.

In another general one or more aspects, a three-dimensional (3D) nanoprobe device includes a body portion comprising a plurality of layers, an extension portion extending from the plurality of layers of the body portion in a first direction to configure nanoprobes, wherein each of the nanoprobes is spaced apart from another in the first direction, and an electrode portion extending from the body portion in a second direction different from the first direction.

Other features and aspects will be apparent from the following detailed description, the drawings, and the claims.

### BRIEF DESCRIPTION OF THE DRAWINGS

FIG. 1A illustrates an example of a cross-section of a three-dimensional (3D) nanoprobe device, according to one or more embodiments.

FIG. 1B illustrates another example of a cross-section of a 3D nanoprobe device, according to one or more embodiments.

FIG. 2A illustrates an example of a cross-section of a body portion of a 3D nanoprobe device, according to one or more embodiments.

FIG. 2B illustrates an example of a cross-section of an extension portion of a 3D nanoprobe device, according to one or more embodiments.

FIG. 2C illustrates another example of a cross-section of an extension portion of a 3D nanoprobe device, according to one or more embodiments.

FIG. 3 illustrates an example of a nanoprobe of a 3D nanoprobe device, according to one or more embodiments.

FIG. 4 illustrates an example of a method of manufacturing a 3D nanoprobe device, according to one or more embodiments.

FIG. 5 illustrates a cross-section of an extension portion of a 3D nanoprobe device to describe a method of manufacturing a 3D nanoprobe device, according to one or more embodiments.

FIG. 6 illustrates another example of a method of manufacturing a 3D nanoprobe device, according to one or more embodiments.

FIG. 7 illustrates a cross-section of an extension portion of a 3D nanoprobe device to describe a method of manufacturing a 3D nanoprobe device, according to one or more embodiments.

Throughout the drawings and the detailed description, unless otherwise described or provided, the same drawing reference numerals may be understood to refer to the same or like elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences within and/or of operations described herein are merely examples, and are not limited to those set forth herein, but may be changed as will be apparent after an understanding of the disclosure of this application, except for sequences within and/or of operations necessarily occurring in a certain order. As another example, the sequences of and/or within operations may be performed in parallel, except for at least a portion of sequences of and/or within operations necessarily occurring in an order, e.g., a certain order. Also, descriptions of features that are known after an understanding of the disclosure of this application may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application. The use of the term “may” herein with respect to an example or embodiment, e.g., as to what an example or embodiment may include or implement, means that at least one example or embodiment exists where such a feature is included or implemented, while all examples are not limited thereto.

Throughout the specification, when a component or element is described as being “on”, “connected to,” “coupled to,” or “joined to” another component, element, or layer it may be directly (e.g., in contact with the other component or element) “on”, “connected to,” “coupled to,” or “joined to” the other component, element, or layer or there may reasonably be one or more other components, elements, layers intervening therebetween. When a component or element is described as being “directly on”, “directly connected to,” “directly coupled to,” or “directly joined” to another component or element, there can be no other elements intervening therebetween. Likewise, expressions, for example, “between” and “immediately between” and “adjacent to” and “immediately adjacent to” may also be construed as described in the foregoing.

Although terms such as “first,” “second,” and “third”, or A, B, (a), (b), and the like may be used herein to describe various members, components, regions, layers, or sections, these members, components, regions, layers, or sections are not to be limited by these terms. Each of these terminologies is not used to define an essence, order, or sequence of corresponding members, components, regions, layers, or sections, for example, but used merely to distinguish the corresponding members, components, regions, layers, or sections from other members, components, regions, layers, or sections. Thus, a first member, component, region, layer, or section referred to in the examples described herein may also be referred to as a second member, component, region, layer, or section without departing from the teachings of the examples.

The terminology used herein is for describing various examples only and is not to be used to limit the disclosure. The articles “a,” “an,” and “the” are intended to include the plural forms as well, unless the context clearly indicates otherwise. As non-limiting examples, terms “comprise” or “comprises,” “include” or “includes,” and “have” or “has” specify the presence of stated features, numbers, operations, members, elements, and/or combinations thereof, but do not preclude the presence or addition of one or more other features, numbers, operations, members, elements, and/or combinations thereof, or the alternate presence of an alternative stated features, numbers, operations, members, elements, and/or combinations thereof. Additionally, while one embodiment may set forth such terms “comprise” or “comprises,” “include” or “includes,” and “have” or “has” specify the presence of stated features, numbers, operations, members, elements, and/or combinations thereof, other embodiments may exist where one or more of the stated features, numbers, operations, members, elements, and/or combinations thereof are not present.

Unless otherwise defined, all terms including technical and scientific terms, used herein have the same meaning as commonly understood by one of ordinary skill in the art to which this disclosure pertains specifically in the context on an understanding of the disclosure of the present application. Terms, such as those defined in commonly used dictionaries, are to be interpreted as having a meaning that is consistent with their meaning in the context of the relevant art and the present disclosure, and are not to be interpreted in an idealized or overly formal sense unless expressly so defined herein.

As used herein, the term “and/or” includes any one and any combination of any two or more of the associated listed items. The phrases “at least one of A, B, and C”, “at least one of A, B, or C’, and the like are intended to have disjunctive meanings, and these phrases “at least one of A, B, and C”, “at least one of A, B, or C’, and the like also include examples where there may be one or more of each of A, B, and/or C (e.g., any combination of one or more of each of A, B, and C), unless the corresponding description and embodiment necessitates such listings (e.g., “at least one of A, B, and C”) to be interpreted to have a conjunctive meaning.

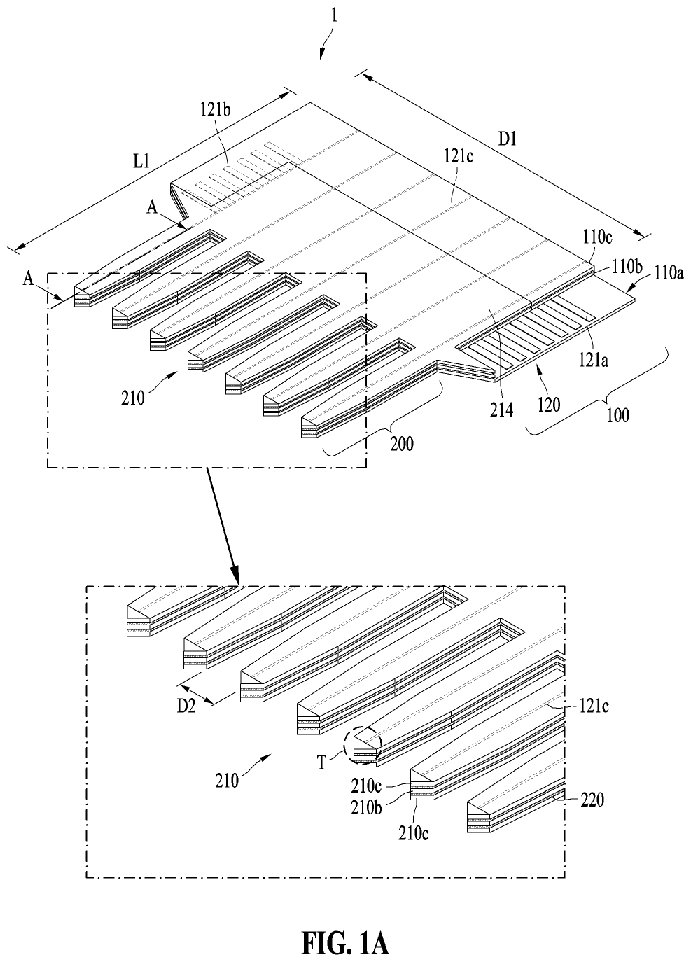

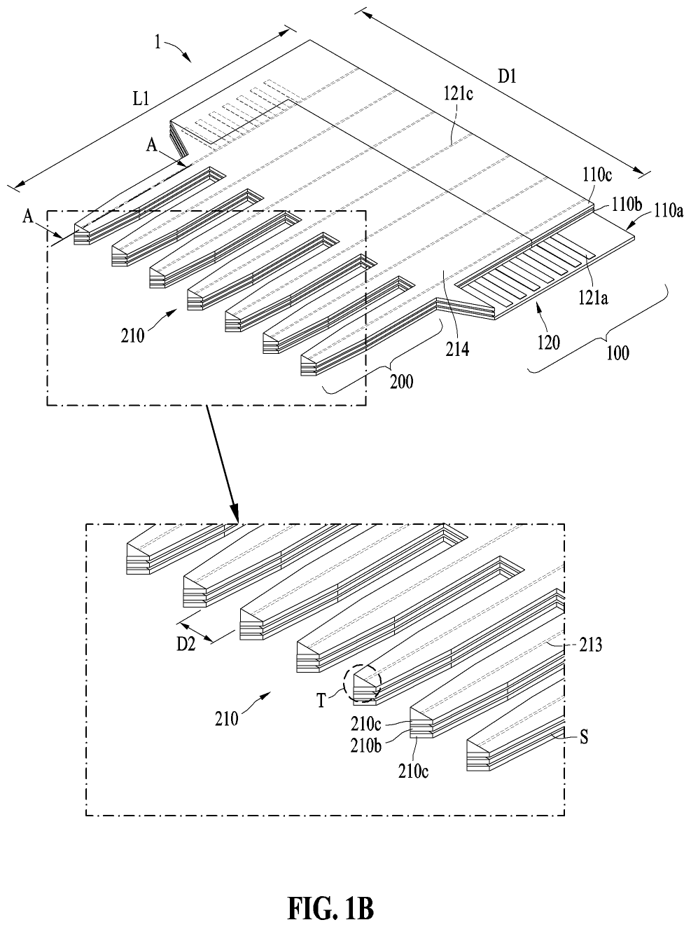

FIG. 1A illustrates an example of a cross-section of a three-dimensional (3D) nanoprobe device, according to one or more embodiments. FIG. 1B illustrates another example of a cross-section of a 3D nanoprobe device, according to one or more embodiments.

Referring to FIGS. 1A and 1B, a 3D nanoprobe device 1 with a 3D structure may be a stack structure in which two-dimensional (2D) planes (e.g., films, thin films, or layers) are stacked. The 3D nanoprobe device 1 may include a region (e.g., an extension portion 200) in which a plurality of nanoprobes 210 with a 3D structure is aligned or arranged in the stack structure. The 3D structure may be formed by a 2D plane lamination method.

In an example, the 3D nanoprobe device 1 may include a body portion 100 and the extension portion 200.

In an example, the body portion 100 may be a stack structure in which a plurality of layers (e.g., layers 110a, 110b, and 110c of FIGS. 1A and 1B) is stacked, and may include an electrode portion 120 for a flow of electricity in at least a part of the body portion 100. In a non-limiting example, the plurality of layers in the stack structure may include at least two layers, at least three layers, or at least five stacked layers. In another example, in the stack structure, the plurality of layers may be stacked so that layers (e.g., layer 110a, 110b, or 110c of FIGS. 1A and 1B) are stacked on each layer (e.g., on a surface). In another example, the stack structure may include an electrode pattern (e.g., electrode patterns 121a, 121b, and 121c of FIGS. 1A and 1B) connected to the electrode portion 120 in each layer (e.g., the layers 110a, 110b, and 110c of FIGS. 1A and 1B) or on each layer (e.g., on a surface).

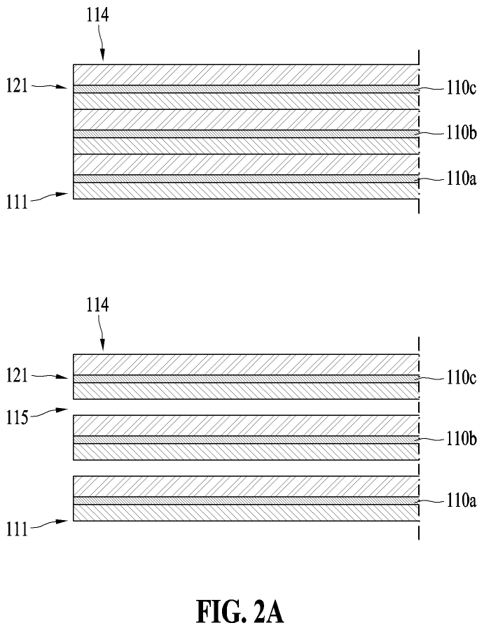

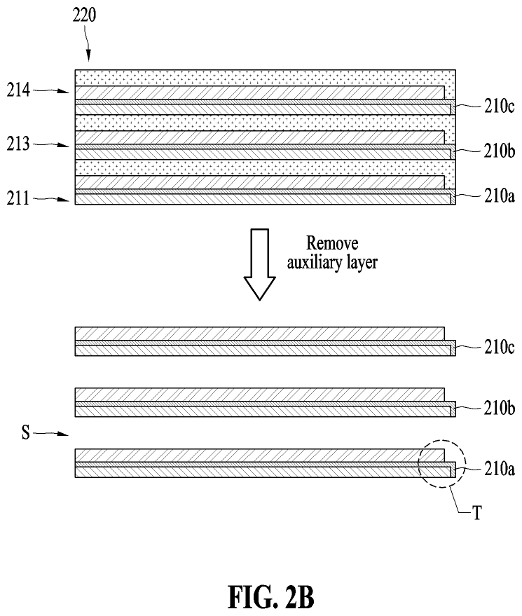

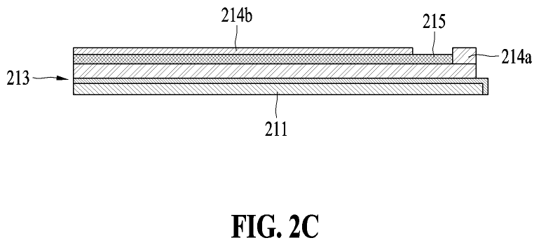

FIG. 2A illustrates a cross-section of the body portion 100 of the 3D nanoprobe device 1. In FIG. 2A, each layer (e.g., the layers 110a, 110b, and 110c of FIGS. 1A and 1B) of the body portion 100 may include a base layer 111, an electrode pattern layer 121, and a protective layer 114.

In an example, the base layer 111 may include a rigid substrate, a flexible substrate, or a flexible and stretchable substrate. In an example, the base layer 111 may be properly selected according to use of the 3D nanoprobe device 1. For example, the base layer 111 may be a substrate that has biocompatibility or relatively low toxicity, or that does not have toxicity.

In an example, in the base layer 111, a substrate may be a substrate including either one or both of an organic material and an inorganic material. The substrate may include, but is not limited to, for example, any one or any combination of any two or more of polyurethane (TPU), silicone rubber (e.g., polydimethylsiloxane (PDMS)) or polymethylphenylsiloxane (PMPS), polyimide, polyethylene isophthalate, cellulose, triacetylcellulose (TAC), polymethyl methacrylate (PMMA), polyvinyl alcohol (PVA), polycarbonate (PC), polyethylene (PE), polypropylene (PP), polystyrene (PS), polyethersulfone (PES), polyethylene naphthalate (PEN), polyethylene terephthalate (PET), polyacrylonitrile (PAN), a silicon substrate (e.g., a monocrystalline Si (c-Si) wafer, a multi-crystalline Si (mc-Si) wafer, and a poly-Si wafer), an alloy (e.g., stainless steel), a carbon-based material (e.g., parylene C, graphite, graphene, silicon carbide, etc.), an oxide (e.g., alumina (Al₂O₃), and silica (SiO₂)), a semiconductor substrate (e.g., a III-V group-based compound semiconductor), an epoxy-based resin (e.g., an epoxy-based photocurable resin) (e.g., commercially available SU-8) (e.g., bisphenol A, bisphenol F, cresol-novolac type epoxy resins, dicyclopentadiene type epoxy resins, trisphenylmethane type epoxy resins, naphthalene-based epoxy resins, or biphenyl type epoxy resins).

In an example, the electrode pattern layer 121 may be formed on the base layer 111. The electrode patterns (e.g., the electrode patterns 121a, 121b, and 121c of FIGS. 1A and 1B) in the electrode pattern layer 121 may include different types of materials capable of enabling the flow of electricity without limitations. For example, an electrode pattern may include any one or any combination of any two or more of a conductive polymer, a conductive oxide, a conductive metal, or a conductive alloy, but is not limited thereto. For example, the electrode pattern may include any one or any combination of any two or more of ITO, IGO, indium zinc oxide (IZO), indium gallium zinc oxide (IGZO), ZnO, ZnO₂, TiO₂, platinum, gold, silver, aluminum, iron, copper, carbon nanotube (CNT), graphene, poly(3,4-ethylenedioxythiophene) (PEDOT):poly(styrenesulfonate) (PSS), polyacetylene, polythiophene (PT), polypyrrole, polyparaphenylene (PPV), polyaniline, polysulfur nitride, stainless steel, such as SUS 304, SUS 316, and SUS 316L, a Co—Cr alloy, a Ti alloy, or nitinol (Ni—Ti), but is not limited thereto.

In an example, the protective layer 114 may protect the electrode pattern layer 121 by blocking moisture from the electrode pattern of the electrode pattern layer 121 and preventing corrosion, and the like. The protective layer 114 may be formed on the electrode pattern layer 121 to cover the electrode pattern layer 121. For example, the electrode pattern layer 121 may be embedded in the protective layer 114. In an example, the protective layer 114 may be formed on at least a portion of each of the layers (e.g., the layers 110a, 110b, and 110c of FIGS. 1A and 1B). In another example, the protective layer 114 may be continuous with a protective layer (e.g., protective layer 214 of FIGS. 1A and 1B) of the extension portion 200.

The protective layer 114 may include an electrical insulating material or a hydrophobic material including either one or both of an inorganic material and an organic material. For example, the protective layer 114 may include an organic dielectric material, such as parylene-C, polyimide, benzocyclobutene (BCB), polybenz oxazole (PBO), bismaleimide triazine (BT), polyarylene ether, fluorinated amorphous carbon (FAC), or the like, and an inorganic dielectric material, such as siloxane, silicone, SiO₂, Si₃N₄, polysilsesquioxane, methyl silane, or the like, but is not limited thereto.

In a non-limiting example, the base layer 111, the electrode pattern layer 121, and the protective layer 114 in the body portion 100 may each have a thickness selected from a range of about 10 nm to about 200 μm; about 10 nm to about 150 μm; about 10 nm to about 120 μm; about 10 nm to about 100 μm; about 10 nm to about 80 μm; about 10 nm to about 50 μm; about 10 nm to about 40 μm; about 10 nm to about 20 μm; about 10 nm to 10 μm; about 10 nm to about 5 μm; about 10 nm to about 2 μm; about 10 nm to about 1 μm; about 10 nm to about 0.8 μm; about 10 nm to about 0.5 μm; or about 10 nm to about 0.1 μm. For example, the base layer 111 may have a thickness of about 10 nm to about 1 μm, and the protective layer 114 may have a thickness of about 10 nm to about 200 μm.

In a non-limiting example, the layers (e.g., the layers 110a, 110b, and 110c of FIGS. 1A and 1B) of the body portion 100 may each have a thickness selected from a range of about 10 nm to about 200 μm; about 20 nm to about 200 μm; about 30 nm to about 200 μm; about 50 nm to about 200 μm; about 100 nm to about 200 μm; about 500 nm to about 200 μm; about 800 nm to about 200 μm; about 1000 nm to about 200 μm; about 1000 nm to about 100 μm; about 1000 nm to about 50 μm; or about 1000 nm to about 10 μm.

In a top part of FIG. 2A, each of the layers (e.g., the layers 110a, 110b, and 110c of FIGS. 1A and 1B) of the body portion 100 may be in contact with a neighboring layer. For example, a base layer of an upper layer may be in contact with an auxiliary layer of a lower layer. In a bottom part of FIG. 2A, each of the layers (e.g., the layers 110a, 110b, and 110c of FIGS. 1A and 1B) of the body portion 100 may further include a support layer 115. The support layer 115 may include either one or both of an organic material and an inorganic material. All materials that may not hinder a function of the body portion 100 or the 3D nanoprobe device 1, or that may fix or support the body portion 100, may be applied without limitations. In an example, the support layer 115 may include the polymers, resins, or water-soluble polymers (e.g., the polymers, resins, or water-soluble polymers mentioned in association with the base layer 111 and the extension portion 200) mentioned herein and may fix or support each layer. In another example, the support layer 115 may include an adhesive resin or a water-soluble polymer and may fix or support each layer.

In an example, the electrode portion 120 may control a flow of electricity to operate the 3D nanoprobe device 1 and may be connected to an external terminal. The electrode portion 120 may be formed in at least a part of the body portion 100. In an example, the electrode portion 120 may be formed in a plurality of regions of the body portion 100. In another example, the body portion 100 may be configured by the electrode pattern layer 121 and may be designed variously according to a shape of the electrode pattern of the electrode pattern layer 121. In another example, the electrode portion 120 may be partially exposed in the protective layer 114 of each of the layers (e.g., the layers 110a, 110b, and 110c of FIGS. 1A and 1B).

The extension portion 200 may be a nanoprobe region that extends from the body portion 100 and includes a plurality of nanoprobes 210. In the extension portion 200, each of the layers (e.g., layers 110a, 110b, and 110c of FIGS. 1A and 1B) of the body portion 100 may be extended and stacked. Layers (e.g., layers 210a, 210b, and 210c of FIGS. 1A and 1B) of the extension portion 200 may have a configuration, at least, partially identical to corresponding layers (e.g., the layers 110a, 110b, and 110c of FIGS. 1A and 1B) of the body portion 100. The extension portion 200 may be formed by transforming a portion of a stack structure of the plurality of layers (e.g., the layers 110a, 110b, and 110c of FIGS. 1A and 1B) of the body portion 100 into a shape of a nanoprobe.

The extension portion 200 may extend in at least one direction of the body portion 100. In an example, the extension portion 200 may extend in a longitudinal direction (e.g., a direction L1 of FIGS. 1A and 1B). In an example, in the extension portion 200, a plurality of layers (e.g., the layers 210a, 210b and 210c of FIGS. 1A and 1B), including a plurality of nanoprobes extending in the longitudinal direction (e.g., the direction L1 of FIGS. 1A and 1B), may be stacked. In an example, the extension portion 200 may extend from one surface, two surfaces, or three surfaces of the body portion 100.

The layers (e.g., the layers 210a, 210b, and 210c of FIGS. 1A and 1B) of the extension portion 200 may include the same or different nanoprobes 210 from each other. In an example, the layers may be different from each other in size (e.g., at least one selected from among a width, a length, a thickness, or a combination thereof), aspect ratio, or arrangement spacing (e.g., D2 of FIGS. 1A and 1B) of the nanoprobes 210.

The nanoprobes 210 of the layers of the extension portion 200 may be arranged in a width direction (e.g., a direction D1 of FIGS. 1A and 1B) (e.g., a transverse direction) of the body portion 100. In a non-limiting example, the nanoprobes 210 may be arranged at a spacing (e.g., D2 of FIGS. 1A and 1B) of about 10 nm to about 100 mm; about 10 nm to about 90 mm; about 10 nm to about 80 mm; about 10 nm to about 60 mm; about 10 nm to about 50 mm; about 10 nm to about 40 mm; about 10 nm to about 30 mm; about 10 nm to about 20 mm; about 10 nm to about 10 mm; about 10 nm to about 1 mm; about 10 nm to about 0.9 mm; about 10 nm to about 0.7 mm; about 10 nm to about 0.5 mm; about 10 nm to about 0.1 mm; about 100 nm to about 100 mm; about 500 nm to about 100 mm; about 800 nm to about 100 mm; about 1000 nm to about 10 mm; or about 4000 nm to about 1 mm.

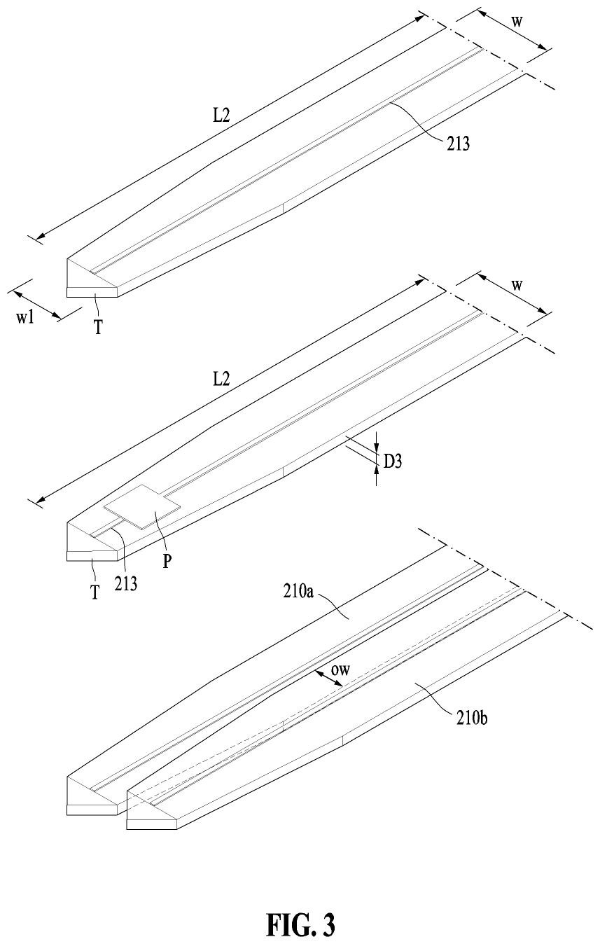

FIG. 3 illustrates an example of a nanoprobe of a 3D nanoprobe device according to one or more embodiments. In FIG. 3, in a non-limiting example, a nanoprobe 210 may have at least one of a thickness D3 of about 10 nm to about 200 μm, a width W of about 10 nm to about 100 mm, a length L2 of about 10 nm to about 100 mm, or a combination thereof. The above-described numerical values may refer to a maximum value, a minimum value, or an average value. In a non-limiting example, a tip region T of the nanoprobe 210 may have a width W1 of about 10 nm to about 100 mm. In a non-limiting example, the nanoprobe 210 may have the thickness D3 of about 10 nm to about 200 μm, the width W of about 10 nm to about 100 mm, and the length L2 of about 10 nm to about 100 mm.

In a non-limiting example, each of the width W1, the width W, and the length L2 may be selected from a range of about 10 nm to about 100 mm; about 10 nm to about 90 mm; about 10 nm to about 80 mm; about 10 nm to about 60 mm; about 10 nm to about 50 mm; about 10 nm to about 40 mm; about 10 nm to about 30 mm; about 10 nm to about 20 mm; about 10 nm to about 10 mm; about 10 nm to about 1 mm; about 10 nm to about 0.9 mm; about 10 nm to about 0.7 mm; about 10 nm to about 0.5 mm; about 10 nm to about 0.1 mm; about 10 nm to about 0.01 mm; about 100 nm to about 100 mm; about 500 nm to about 100 mm; about 800 nm to about 100 mm; about 1000 nm to about 10 mm; or about 4000 nm to about 1 mm.

The nanoprobe 210 may have an aspect ratio of about 0.000005 or greater; from about 0.000005 to about 200000; from about 0.00005 to about 200000; from about 0.0005 to about 200000; from about 0.005 to about 200000; from about 0.05 to about 200000; from about 0.5 to about 200000; from about 1 to about 200000; from about 0.000005 to about 100000; from about 0.000005 to about 10000; from about 0.000005 to about 1000; and from about 0.000005 to about 100. In the examples, the aspect ratio may be between the length L2 and the width W or W1 of the nanoprobe 210.

In an example, the thickness of the nanoprobe 210 may be constantly reduced in a direction from the body portion 100 to the tip region T. In another example, the nanoprobe 210 may have a shape of a needle having a width constantly decreasing from the body portion 100 to the tip region T. This may be controlled by the aspect ratio.

In an example, the nanoprobe 210 may include the tip region T (e.g., T of FIGS. 1A and 1B). The tip region T may include the same or different conductive material as or from that of the electrode pattern. In an example, the tip region T may include any one or any combination of any two or more of ITO, IGO, indium zinc oxide (IZO), indium gallium zinc oxide (IGZO), ZnO, ZnO₂, TiO₂, platinum, gold, silver, aluminum, iron, copper, carbon nanotube (CNT), graphene, poly(3,4-ethylenedioxythiophene) (PEDOT):poly(styrenesulfonate) (PSS), polyacetylene, polythiophene (PT), polypyrrole, polyparaphenylene (PPV), polyaniline, polysulfur nitride, stainless steel, such as SUS 304, SUS 316, and SUS 316L, a Co—Cr alloy, a Ti alloy, or nitinol (Ni—Ti), but is not limited thereto.

The extension portion 200 may include a void or separation region (e.g., separation regions S of FIG. 1B and FIG. 2B) between the layers. In an example, the separation region (e.g., the separation regions S of FIG. 1B and FIG. 2B) may be empty or may include an auxiliary layer (e.g., an auxiliary layer 220 of FIG. 1A). In an example, the separation region S may have a height of about 10 nm to about 30 μm; about 10 nm to about 20 μm; about 10 nm to about 10 μm; about 10 nm to about 8 μm; about 10 nm to about 5 μm; about 10 nm to about 3 μm; about 10 nm to about 2 μm; about 10 nm to about 1 μm; about 50 nm to about 30 μm; about 100 nm to about 30 μm; about 300 nm to about 30 μm; about 500 nm to about 30 μm; or about 800 nm to about 30 μm. In another example, the height of the separation region S may continue to increase toward the tip region T or may remain unchanged. In another example, the separation region (e.g., the separation regions S of FIG. 1B and FIG. 2B) may be empty to the tip region T or from the tip region T to a region adjacent to the tip region T, and at least a portion of the other regions may include the auxiliary layer 220. In an example, the auxiliary layer 220 may include a water-soluble polymer that is removed with water or an enzyme, and may be formed by removing the water-soluble polymer with water or an enzyme in a device including the auxiliary layer 220 in FIGS. 1A and 2B. For example, the 3D nanoprobe device 1 of FIG. 1B may be formed. In an example, a thickness of the auxiliary layer 220 may be equal to the height of the separation region (e.g., the separation regions S of FIG. 1B and FIG. 2B).

The auxiliary layer 220 may include a polymer soluble in water or an enzyme, and may include, for example, a water-soluble polymer. In an example, the water-soluble polymer may include any one or any combination of any two or more of polyvinyl alcohol (PVA), poly(acrylic acid) (PAA), polystyrenesulfonate (PSS), starch, gum (polysaccharide) (e.g., agar, algin, carraginan, Arabia, and Xanthane Rubber), cellulose, a cellulose derivative (e.g., methyl cellulose (MC), ethyl cellulose, hydroxyethyl cellulose (HEC), and carboxymethyl cellulose (CMC)), polyamine, polyethyleneimine (PEI), polyethylene oxide (PEO), poly(4-styrenesulfonic acid) salt (PSSA), polysilicic acid (PsiA), phosphoric acid, phosphate (polyphosphoric acid, PPA), or polyepoxysuccinic acid sodium salt (PESA), but is not limited thereto. In another example, the water-soluble polymer may be any one or any combination of any two or more polyvinyl alcohol (PVA), poly(acrylic acid) (PAA), starch, cellulose, and/or a cellulose derivative.

According to one or more embodiments, the layers of the extension portion 200 may be sequentially stacked such that the nanoprobes 210 may overlap each other. For example, when viewed in a vertical direction, the layers may be stacked such that all nanoprobes (e.g., the nanoprobes 210 of FIGS. 1A and 1B) may overlap each other.

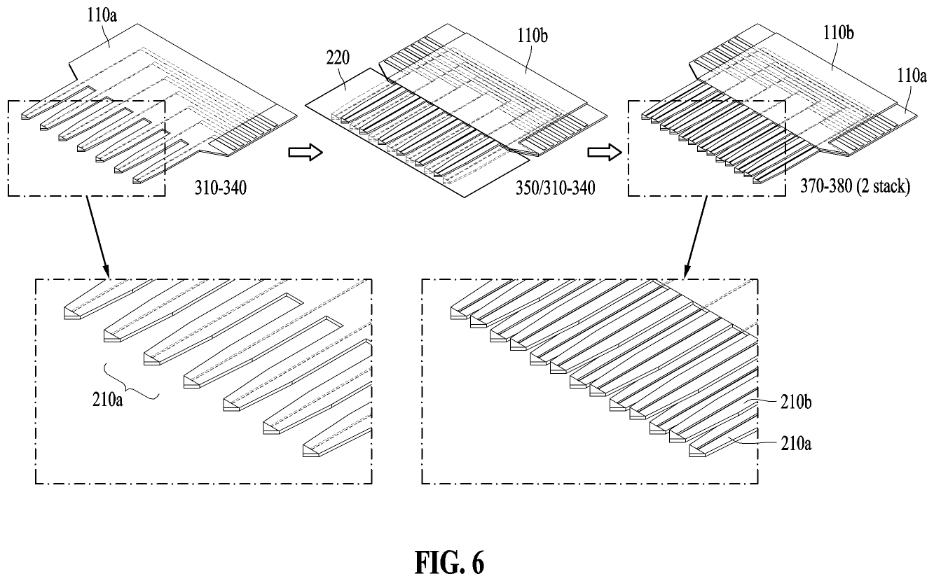

In another example, the layers of the extension portion 200 may be cross-stacked such that nanoprobes (e.g., the layers 210a and 210b of FIGS. 3 and 6) may cross each other. In one example, when viewed in the vertical direction, the layers may be stacked such that the nanoprobes may at least partially overlap each other. In another example, when viewed in the vertical direction, the layers may be stacked such that the nanoprobes may be separated instead of overlapping. In an example, referring to FIG. 3, the layers (e.g., the layers 210a and 210b of FIGS. 3 and 6) in the extension portion 200 may be cross-stacked such that the nanoprobes 210 may overlap each other with an overlap width OW corresponding to about 0% to less than about 100%; about 0% to about 80%; about 0% to about 70%; about 0% to about 50%; about 1% to about 40%; about 5% to about 40%; and about 5% to about 30% of an average width W. For example, when the overlap width OW corresponds to about 0% of the average width W, the layers of the extension portion 200 may be cross-stacked such that the nanoprobes may be free of an overlapping region. When viewed in the vertical direction, the layers may be arranged such that the nanoprobes may be individually separated from each other. In an example, a density (e.g., a high density) of the nanoprobes may be adjusted by adjusting an overlapping region between the nanoprobes 210 in the extension portion 200.

The layers (e.g., the layers 210a, 210b, and 210c of FIGS. 1A and 1B) of the extension portion 200 may each have a thickness of about 10 nm to about 200 μm, which may correspond to the thickness of the nanoprobe 210.

FIG. 2B illustrates an example of a cross-section of an extension portion of a 3D nanoprobe device according to one or more embodiments. In FIG. 2B, each of the layers 210a, 210b, and 210c of the extension portion 200 may include a nanoprobe 210 that includes a base layer 211 having a shape of a nanoprobe, an electrode pattern layer 213 on the base layer 211, and the protective layer 214 on the electrode pattern layer 213. The base layer 211 and the protective layer 214 may extend from the body portion 100, and may have the same configuration as that of the base layer 111 and the protective layer 114 of the body portion 100.

The electrode pattern layer 213 may include an electrode pattern connected to or continuous with the electrode portion 120 of the body portion 100.

The electrode pattern of the electrode pattern layer 213 may have various shapes. For example, the electrode pattern may have any one or any combination of a line, including any one or any combination of any two or more of a straight line, an oblique line, a zigzag, a streamline, a curve, or a comb profile; a shape including at least one of a circle, an ellipse, a doughnut, or a polygon; or a combination thereof. In an example, the line may be a single line or a double line. In another example, the line may be a zigzag curve or a straight line. In an example, the streamline may have a shape of “S” or “Z”. In an example, the polygon may be a triangle, a quadrangle (e.g., a square, a rectangle, etc.), a pentagon, a hexagon, and the like.

The electrode pattern of the electrode pattern layer 213 may have a thickness, a diameter, or a width of about 10 nm to about 1 μm. In an example, the electrode pattern of the electrode pattern layer 213 may include a line with a line thickness of about 10 nm to about 100 mm. In an example, the electrode pattern 213 may include a shape with a diameter or a width of about 10 nm to about 100 mm.

Referring to FIG. 2C, each of the layers of the extension portion 200 may include a nanoprobe that includes a base layer 211 having a shape of a nanoprobe, an electrode pattern layer 213 on the base layer 211, a first protective layer 214a on the electrode pattern layer 213, an electrode pad layer 215, and a second protective layer 214b. The base layer and the protective layer may extend from the body portion 100 and may have the same configuration as that of the base layer 111 of the body portion 100. In an example, the first protective layer 214a and the second protective layer 214b may include the same or different materials. In an example, the first protective layer 214a and the second protective layer 214b may have the same thickness or different thicknesses. For example, the thickness of the second protective layer 214b may be less than that of the first protective layer 214a.

The electrode pad layer 215 may be formed on the first protective layer 214a (e.g., on a top surface). In an example, referring to FIG. 3, the electrode pad layer 215 may include an electrode pad P having a shape of at least one of a circle, an ellipse, a doughnut, a polygon, or a combination thereof. In another example, the electrode pad layer 215 may include an electrode pad P in a region adjacent to the tip region T and may be connected to the electrode portion 120 of the body portion 100 through an electrode line. In an example, the electrode pad P of the electrode pad layer 215 may apply an external signal. In an example, electrochemical signal or electric signal recording or electrical stimulation and measurement may be simultaneously performed.

The tip region T of the nanoprobe may be exposed in the first protective layer 214a and the second protective layer 214b, and the electrode pad P of the electrode pad layer 215 may be exposed in the second protective layer 214b.

The electrode pad P of the electrode pad layer 215 may include the same or different material from that of the electrode pattern of the electrode pattern layer 213.

The base layer 211, the protective layer 214, the first protective layer 214a, and the second protective layer 214b may each have a thickness of about 10 nm to about 200 μm.

According to an example, an electronic device may include a 3D nanoprobe device (e.g., the 3D nanoprobe device 1 of FIGS. 1A and 1B).

The 3D nanoprobe device of the electronic device may be an electrode for sensing a signal or a target material, or applying a signal. In an example, the 3D nanoprobe device of the electronic device may include nanoprobes with a high aspect ratio or a high degree of integration. In an example, the 3D nanoprobe device of the electronic device may be inserted or implanted into a living body. In an example, the 3D nanoprobe device of the electronic device may provide a high sensitivity while minimizing damage to a living body.

The electronic device may measure either one or both of a biosignal and a biomaterial. In an example, the electronic device may be a biosensor for measuring an electric signal or detecting a target material in vitro or in vivo. In another example, the electronic device may be a biosensor for intracellular recording by measuring an electric signal. In another example, the electronic device may measure and record an electric signal in vitro or in vivo. In another example, the electronic device may be a device for intracellular recording by measuring and recording an electric signal in a brain region. In another example, the electronic device may apply an external stimulus in vitro or in vivo. For example, the electronic device may apply an electrical stimulus or a stimulus through external light to a brain region (e.g., a cranial nerve). For example, an electrical stimulus or a stimulus through external light may be applied to a brain region (e.g., a cranial nerve), and an electric signal of a brain may be measured.

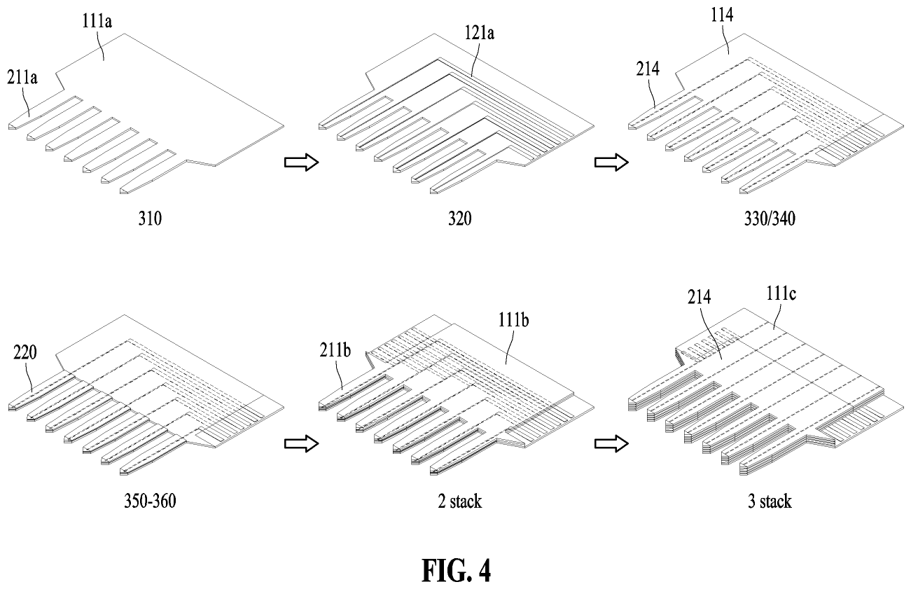

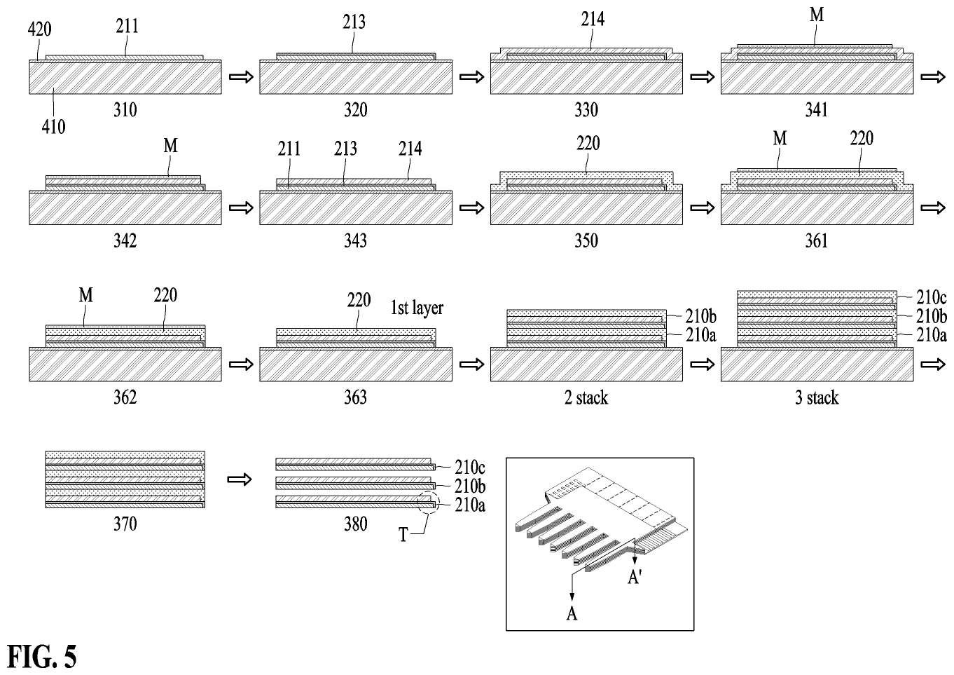

FIG. 4 illustrates an example of a method of manufacturing a 3D nanoprobe device according to one or more embodiments. FIG. 5 illustrates an example of a process related to the extension portion 200 in a method of manufacturing a 3D nanoprobe device according to one or more embodiments. FIG. 5 illustrates a cross-section of the extension portion 200 taken along a line indicated by A in FIGS. 1A and 1B.

Referring to FIG. 4, the method may include an operation 310 of preparing a base layer, operation 320 of forming an electrode pattern layer, and operation 330 of forming a protective layer.

In FIG. 4, the method may include a process of stacking a plurality of layers to form a stack structure. In an example, the method of FIG. 4 may include a 1-stack lamination process of one stack (e.g., layer 110a, 110b, or 110c), which includes operation 310 of preparing a base layer, operation 320 of forming an electrode pattern layer, operation 330 of forming a protective layer, and operation 340 of etching the protective layer. The 1-stack lamination process may be repeatedly performed “n” times to provide a 3D nanoprobe device in which “n” stacks are stacked. After the 1-stack lamination process, a lamination process may be repeatedly performed by applying operation 350 to form an auxiliary layer on the one stack (e.g., on a surface of an outermost layer). For example, the 1-stack lamination process, including operations 310 through 340, and operation 350, may be repeatedly performed to manufacture a 3D nanoprobe device (e.g., the 3D nanoprobe device 1 of FIGS. 1A and 1B) by a 2D plane lamination process.

Referring to FIGS. 4 and 5, operation 310 may include an operation of forming a sacrificial layer 420 (e.g., any one or any combination of any two or more of Cr/Au/Cr, Cu, Ni, Cr or SiO₂) on a base substrate 410 (e.g., a silicon wafer), and an operation of forming a base layer 211 (e.g., a film or a thin film) patterned to have a shape of a nanoprobe on at least one surface. In an example, the patterned base layer 211 (e.g., a film or a thin film) may be disposed on the sacrificial layer 420, or the base layer 211 may be deposited on the sacrificial layer 420 and may be patterned. In an example, the body portion 100 and the extension portion 200, having a shape of a nanoprobe, may be formed in one base layer 211.

In operation 320, a conductive material may be deposited on the base layer 211 (e.g., a top surface) and patterned into an electrode pattern. In an example, an electrode pattern connected from the body portion 100 to the extension portion 200 may be formed according to a tip region of a nanoprobe and a desired pattern.

In operation 330, the electrode pattern may be covered by depositing or applying a protective layer 214 on at least a portion of the base layer 211 on which the electrode pattern is formed.

Operation 340 may include operation 341 of forming an etching mask M on the protective layer 214, operation 342 of performing etching, and operation 343 of removing the etching mask M. In operation 342, the protective layer 214 corresponding to the tip region of the nanoprobe or a region adjacent to the tip region may be removed. For etching, a dry etching process or a wet etching process may be used. In the dry etching process, etching gas, or plasma (O₂) may be used. An etchant, including an acid, may be used in the wet etching process.

In operation 350, an auxiliary layer 220 may be formed on the protective layer 214 by a deposition or coating process. The auxiliary layer 220 may include a water-soluble polymer. In an example, the auxiliary layer 220 may be formed on at least a portion of the extension portion 200 (e.g., on a nanoprobe).

Operation 360 of etching an auxiliary layer may include operation 361 of forming an etching mask M on the auxiliary layer 220, operation 362 of performing etching, and operation 363 of removing the etching mask M. In operation 362, the auxiliary layer 220 may be etched according to the shape of the 3D nanoprobe device 1.

After operation 360 is performed, a plurality of (e.g., “n”) stacks (e.g., the layers 210a, 210b, and 210c) may be stacked by repeatedly performing “n” times operations 310 through 360. The lamination process may form a stack structure with “n” stacks.

In the lamination process, layers may be sequentially stacked or cross-stacked. FIG. 6 illustrates another example of a method of manufacturing a 3D nanoprobe device according to one or more embodiments. FIG. 6 shows a process of cross-stacking layers in the lamination process. In FIG. 6, to secure a space for cross-stacking on an (n−1)-th stack (e.g., layer 210a), an auxiliary layer 220 with an appropriate size may be formed in operation 350. Subsequently, operation 310 for an n-th stack may be performed instead of performing operation 360 of etching the auxiliary layer. A base layer (e.g., a base layer 211b) of an n-th stack (e.g., the layer 210b) in operation 310 may be aligned or arranged and stacked to cross a nanoprobe of the (n−1)-th stack (e.g., the layer 210a). Subsequently, for the n-th stack, the lamination process of operations 320 through 340, including operations 341, 342, and 343, may be performed, as shown in FIGS. 4 and 5. If desired, operation 360 of etching the auxiliary layer 220 may be additionally performed to adjust the structure or shape of the auxiliary layer 220 in the lamination process.

According to an example, operation 370 of separating a stack structure from a base substrate may be included. In operation 370, a stack structure may be separated from the base substrate 410 by removing the sacrificial layer 420 when the lamination process is completed.

According to an example, operation 380 of removing the auxiliary layer may be included. In operation 380, the auxiliary layer 220 may be removed from the stack structure when operation 370 is performed. The auxiliary layer 220 may be removed with water, enzymes, sonication, and the like.

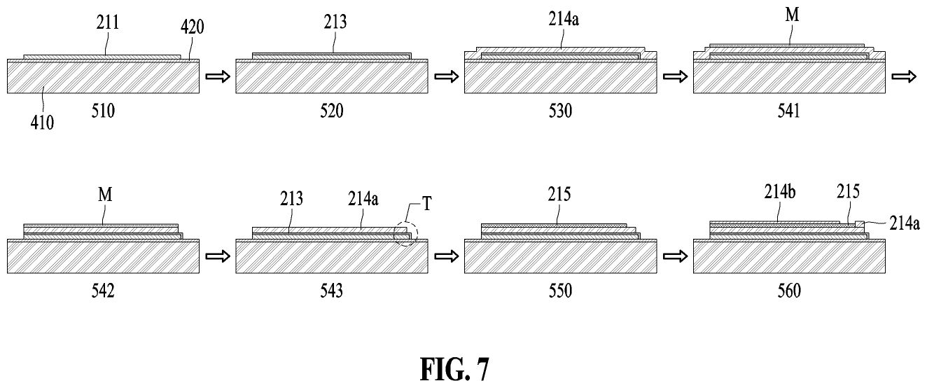

Referring to FIG. 7, a method of manufacturing a 3D nanoprobe device may include operation 510 of preparing a base layer, operation 520 of forming an electrode pattern layer, operation 530 of forming a first protective layer, operation 540 of etching the first protective layer (not shown in FIG. 7), operation 550 of forming an electrode pad 550, and operation 560 of forming a second protective layer. The description provided with reference to FIGS. 4 through 6 may equally apply to operations 510 through 540. In operation 550, a conductive material may be deposited and patterning may be performed. The electrode pad may be patterned in a portion of a nanoprobe region. For example, the electrode pad may be patterned to include an electrode line to connect the electrode pad to the electrode portion of the body portion 100.

Operation 540 may include operation 541 of forming an etching mask M on the first protective layer 214a, operation 542 of performing etching, and operation 543 of removing the etching mask M. In operation 542, the first protective layer 214a corresponding to a tip region of the nanoprobe or a region adjacent to the tip region may be removed. For etching, a dry etching process or a wet etching process may be used. In the dry etching process, etching gas, or plasma (O₂) may be used. An etchant, including an acid, may be used in the wet etching process.

In operation 550, the conductive material may be deposited on the first protective layer 214a, and patterning may be performed to have a desired shape.

In operation 560, the electrode pad P and the tip region T may be patterned to be exposed when the second passivation layer 214b is formed. In an example, the second protective layer 214b formed in operation 560 and the first protective layer 214a formed in operation 530 may be formed of the same material or different materials.

To perform a lamination process, the method of FIG. 7 may include, after operation 560, an operation (not shown in FIG. 7) of forming an auxiliary layer. The lamination process may be performed as described above with reference to FIGS. 4 through 6.

In the present disclosure, a deposition may be performed using a deposition process known in the art to which the present disclosure belongs. For example, a typical deposition process known in the art to which the present disclosure belongs, such as sputtering, thermal evaporation, electron-beam (E-beam) evaporation, atomic layer deposition, chemical vapor deposition (CVD), low-pressure chemical vapor deposition, plasma enhanced chemical vapor deposition, and/or thermal oxidation deposition method, may be used.

In the present disclosure, a coating may be performed using a typical coating process known in the art to which the present disclosure belongs, for example, spin coating, roll coating, spray coating, dip coating, flow coating, doctor blade, dispensing, and inkjet printing; however, examples are not limited thereto.

In the present disclosure, wet etching or dry etching may be used. An etchant, including an acid, may be used in the wet etching. In the dry etching, etching gas, or plasma (e.g., O₂ plasma etching) may be used.

In the present disclosure, as a patterning process, a patterning process, known in the art to which the present disclosure belongs, a photomask, etching, and the like may be used, which are not described in detail herein.

In the case of nanoscale electrodes developed according to a related art, it is difficult to manufacture an electrode with a large area on a wafer scale, and it is also difficult to form a structure with a high aspect ratio because electrodes are manufactured with a vertical structure. In developed nanoscale electrodes, electrodes standing in a process after a structure fabrication process may collapse or cause damage to a structure, which may reduce the yield and efficiency of a manufacturing process.

To solve the above problems, examples may provide a 3D nanoprobe device (e.g., the 3D nanoprobe device 1 of FIGS. 1A and 1B) acquired by a lamination process of 2D planes (e.g., films or thin films).

According to examples, the 3D nanoprobe device (e.g., the 3D nanoprobe device 1 of FIGS. 1A and 1B) may implement the integration of nanoprobes (e.g., nanoprobe electrodes) that are properly arranged with a high density.

According to examples, in the 3D nanoprobe device (e.g., the 3D nanoprobe device 1 of FIGS. 1A and 1B), electrodes may be stacked and arranged in a lamination method in a 2D plane. Thus, it may be possible to precisely and accurately align an arrangement of nanoprobes (e.g., the nanoprobes 210 (e.g., nanoprobe electrodes) of FIGS. 1A and 1B) in a desired shape or position.

According to examples, in the 3D nanoprobe device (e.g., the 3D nanoprobe device 1 of FIGS. 1A and 1B), an integration of nanoprobes (e.g., nanoprobe electrodes) may be possible using a multilayer lamination method in the 2D plane. Thus, it may be possible to integrate nanoprobes (e.g., nanoprobe electrodes) with a high density, and adjust a tip size (e.g., a diameter) of a nanoprobe from a few nanometers to tens of millimeters and a length of the nanoprobe (that is not limited). In addition, it may be possible to implement nanoprobes (e.g., nanoprobe electrodes) having a high aspect ratio.

According to examples, the 3D nanoprobe device (e.g., the 3D nanoprobe device 1 of FIGS. 1A and 1B) may be inserted or implanted into a human body. For example, the 3D nanoprobe device may be inserted deeply into a brain region with a target position inside the human body and may be applied in vivo.

According to examples, it may be possible to increase the production yield and process efficiency of the 3D nanoprobe device (e.g., the 3D nanoprobe device 1 of FIGS. 1A and 1B) using a multilayer lamination process of 2D planes (e.g., films or thin films).

According to examples, the 3D nanoprobe device (e.g., the 3D nanoprobe device 1 of FIGS. 1A and 1B) may be implanted or inserted into a human body and have structural and functional stability.

According to an example, a 3D nanoprobe device (e.g., the 3D nanoprobe device 1 of FIGS. 1A and 1B) may include a body portion 100 in which a plurality of layers is stacked, an extension portion 200 which extends from the body portion 100 in a longitudinal direction L1 and in which a plurality of layers having a plurality of nanoprobes 210 is stacked, and an electrode portion 120 formed on at least a part of the body portion 100.

According to an example, the layers in the extension portion 200 may extend from corresponding layers of the body portion 100, respectively. The nanoprobes 210 of the layers in the extension portion 200 may be continuous with corresponding layers of the body portion 100 and may be arranged in a width direction D1 of the body portion 100.

According to an example, the plurality of nanoprobes 210 may be arranged at a spacing of about 10 nm to about 100 mm in the width direction D1 of the body portion 100.

According to an example, the plurality of nanoprobes 210 may have at least one of a thickness of about 10 nm to about 200 μm, a width of about 10 nm to about 100 mm, a length of about 10 nm to about 100 mm, or a combination thereof.

According to an example, the nanoprobe 210 may have an aspect ratio of about 0.000005 to about 200000.

According to an example, the extension portion 200 may include a separation region S between the layers. The separation region S may be empty, or may include a water-soluble polymer.

According to an example, the separation region S of the extension portion 200 may be empty to a tip region T of a nanoprobe or from the tip region T to a region adjacent to the tip region T, and may include a water-soluble polymer in at least a part of the other regions.

According to an example, the separation region S may have a height of about 10 nm to about 30 μm.

According to an example, the layers in the extension portion 200 may be sequentially stacked such that the nanoprobes 210 may overlap each other, or the layers in the extension portion 200 may be cross-stacked such that the nanoprobes 210 may cross each other.

According to an example, the layers in the extension portion 200 may be cross-stacked, so the nanoprobes 210 may be free of an overlapping region.

According to an example, the layers in the extension portion 200 may be cross-stacked such that the nanoprobes 210 may overlap each other with an overlap width OW corresponding to 0% to less than 100% of an average width of the nanoprobes 210.

According to an example, each of the layers of the extension portion 200 may include a nanoprobe 210 that includes a base layer 211 with a nanoprobe shape, an electrode pattern layer 213 on the base layer 211; and a protective layer 214 on the electrode pattern layer 213. The base layer 211 may extend from the body portion 100, and a tip region T of the nanoprobe may be exposed in the protective layer 214.

According to an example, each of the layers of the extension portion 200 may include a nanoprobe 210 that includes a base layer 211 having a shape of a nanoprobe, an electrode pattern layer 213 on the base layer 211, a first protective layer 214a on at least a portion of the electrode pattern layer 213, an electrode pad layer 215 on at least a portion of the first protective layer 214a, and a second protective layer 214b on at least a portion of the electrode pad layer 215. The base layer 211 may extend from the body portion 100. a tip region T of a nanoprobe may be exposed in the first protective layer 214a and the second protective layer 214b, and an electrode pad P of the electrode pad layer 215 may be exposed in the second protective layer 214b. In an example, the tip region T may be exposed in the first protective layer 214a and the second protective layer 214b.

According to an example, the base layer 211, the protective layer 214, the first protective layer 214a, and the second protective layer 214b may each have a thickness of about 10 nm to about 200 μm.

According to an example, the electrode pattern may include any one or any combination of any two or more of a straight line, an oblique line, a zigzag, a streamline, a curve, a comb profile, or a combination thereof. The electrode pad may have the shape of any one or any combination of any two or more of a circle, an ellipse, a doughnut, a polygon, or a combination thereof.

According to an example, an electronic device may include a 3D nanoprobe device according to examples.

According to an example, the electronic device may be configured to measure either one or both of a biosignal and a biomaterial.

According to an example, the electronic device may be configured to perform any one or any combination of any two or more of measuring an electrochemical signal, measuring an electric signal, or applying an external stimulus in vitro or in vivo.

According to an example, the electronic device may be configured to measure a brain signal.

According to an example, the electronic device may be configured to apply an external stimulus to a living body.

According to an example, the electronic device may be inserted into a living body.

In an example, at least a portion of the electronic device may be inserted. In an example, at least a portion or all portions of a nanoprobe region (e.g., the extension portion 200 of FIGS. 1A and 1B) of the 3D nanoprobe device of the electronic device may be inserted.

According to an example, the electronic device may be implanted into a living body.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, in addition to the above and all drawing disclosures, the scope of the disclosure is also inclusive of the claims and their equivalents, i.e., all variations within the scope of the claims and their equivalents are to be construed as being included in the disclosure.

What is claimed is:

1. A three-dimensional (3D) nanoprobe device comprising:
a body portion comprising a plurality of body layers that are stacked;
a plurality of nanoprobes respectively extending longitudinally from the body portion, with each of the plurality of nanoprobes comprising a plurality of extension layers that are stacked; and
an electrode portion disposed on a portion of the body portion.

2. The 3D nanoprobe device of claim 1, wherein, for two or more of the plurality of nanoprobes, the plurality of extension layers respectively extend from, and are respectively longitudinally continuous with, the body layers.

3. The 3D nanoprobe device of claim 1, wherein each nanoprobe of the plurality of nanoprobes is spaced apart from a nearest other nanoprobe of the plurality of nanoprobes by about 10 nanometers (nm) to about 100 millimeters (mm) in a transverse direction of the body portion.

4. The 3D nanoprobe device of claim 1, wherein the plurality of nanoprobes has any one or any combination of any two or more of a thickness of about 10 nm to about 200 micrometers (μm), a width of about 10 nm to about 100 mm, or a length of about 0 nm to about 100 mm.

5. The 3D nanoprobe device of claim 1, wherein each of two or more of the plurality of nanoprobes has an aspect ratio of about 0.000005 to about 200000.

6. The 3D nanoprobe device of claim 1, wherein, each of two or more of the plurality of nanoprobes, comprise a separation region respectively between two or more of the plurality of extension layers, and
the separation region is empty or at least a portion of the separation region comprises a water-soluble polymer.

7. The 3D nanoprobe device of claim 6, wherein a portion of the separation region is empty and another portion of the separation region comprises the water-soluble polymer.

8. The 3D nanoprobe device of claim 6, wherein respective tip regions of the two or more of the plurality of nanoprobes are empty and another region of the separation region comprises a water-soluble polymer.

9. The 3D nanoprobe device of claim 6, wherein the separation region has a height of about 10 nm to about 30 μm.

10. The 3D nanoprobe device of claim 1, wherein, for each of two or more of the nanoprobes, respective extension layers, of the two or more of the nanoprobes, are sequentially stacked such that adjacent nanoprobes overlap each other, or
are cross-stacked such that the adjacent nanoprobes cross each other.

11. The 3D nanoprobe device of claim 1, wherein, for each of two or more of the nanoprobes, respective extension layers of adjacent nanoprobes are cross-stacked such that the adjacent nanoprobes are free of overlapping regions.

12. The 3D nanoprobe device of claim 1, wherein, for each of two or more of the nanoprobes, respective extension layers of adjacent nanoprobes are cross-stacked such that the adjacent nanoprobes overlap each other with an overlap width corresponding to 0% to less than 100% of an average width of the nanoprobes.

13. The 3D nanoprobe device of claim 1, wherein each nanoprobe of the plurality of nanoprobes comprise:
a base layer having a shape of the nanoprobe;
an electrode pattern layer disposed on the base layer; and
a protective layer, disposed on the electrode pattern layer, having an exposed tip region,
wherein the base layer extends from the body portion.

14. The 3D nanoprobe device of claim 1, wherein, for each of two or more of the plurality of nanoprobes, the plurality of extension layers comprise:
a base layer having a shape of a corresponding nanoprobe;
an electrode pattern layer disposed on the base layer;
a first protective layer disposed on the electrode pattern layer;
an electrode pad layer disposed on the first protective layer; and
a second protective layer disposed on the electrode pad layer,
wherein the base layer extends from the body portion,
wherein a tip region of the corresponding nanoprobe is exposed in the first protective layer and the second protective layer, and
wherein an electrode pad of the electrode pad layer is exposed in the second protective layer.

15. The 3D nanoprobe device of claim 14, wherein, for each of two or more of the plurality of nanoprobes, each of the base layer, the protective layer, the first protective layer, and the second protective layer has a thickness of about 10 nm to about 200 μm.

16. The 3D nanoprobe device of claim 14, wherein
the electrode pattern layer comprises an electrode pattern comprising any one or any combination of any two or more of a straight line, an oblique line, a zigzag, a streamline, a curve, or a comb profile, and
the electrode pad layer comprises an electrode pad having a shape of any one or any combination of any two or more of a circle, an ellipse, a doughnut, or a polygon.

17. An electronic device comprising a three-dimensional (3D) nanoprobe device, the 3D nanoprobe comprising:
a body portion in which a plurality of body layers is stacked;
an extension portion extending longitudinally from the body portion and in which a plurality of extension layers having a plurality of nanoprobes is stacked; and
an electrode portion disposed on a portion of the body portion.

18. The electronic device of claim 17, wherein the electronic device is configured to measure any one or any combination of any two or more of a biosignal, a biomaterial, and a brain signal.

19. The electronic device of claim 17, wherein the electronic device is configured to apply an external stimulus to a living body or is an implantable device.

20. A three-dimensional (3D) nanoprobe device comprising:
a body portion comprising a plurality of layers;
an extension portion extending from the plurality of layers of the body portion in a first direction to configure nanoprobes, wherein each of the nanoprobes is spaced apart from another in the first direction; and
an electrode portion extending from the body portion in a second direction different from the first direction.
